(19) United States
(12) Patent Application Publication
(10) Pub. No.: US 2019/0343692 A1
(43) Pub. Date: Nov. 14, 2019

(54) ABSORBENT ORGANIC MATERIAL

(71) Applicant: Nia Peters, Saint Albans, NY (US)

(72) Inventor: Nia Peters, Saint Albans, NY (US)

(21) Appl. No.: 16/408,632

(22) Filed: May 10, 2019

Related U.S. Application Data
(60) Provisional application No. 62/670,004, filed on May 11, 2018.

(51) Int. Cl. A61F 13/472; A61F 13/84; A61L 15/20; A61F 13/537

(52) U.S. Cl. CPC A61F 13/472; A61F 13/8405; A61F 13/511; A61F 13/53747; A61F 2013/8408; A61L 15/20

(57) ABSTRACT

An absorbent organic material includes a non-permeable organic bottom layer. The bottom layer includes an upper and a lower surface. At least one core layer is disposed on the upper surface of the bottom layer. The at least one core layer includes a blend of degummed hemp fiber and a super absorbent polymer. An outermost layer includes a permeable composite of organic cotton and hemp fibers disposed on the at least one core layer.

### CROSS-REFERENCE TO RELATED APPLICATION

This U.S. claims priority to U.S. Provisional Patent Application No. 62/670,004, filed on May 11, 2018, the disclosure of which is incorporated by reference herein in its entirety.

### TECHNICAL FIELD

The present disclosure relates to an absorbent material, and more particularly to an absorbent organic material applicable to feminine hygiene products.

### BACKGROUND

Conventional feminine hygiene products may include non-organic pesticide-treated cotton, rayon, as well as a myriad of other chemicals. For example, materials, such as cotton, included in feminine hygiene products such as pads and tampons may be bleached, thus producing dioxins associated with endometriosis and cancer. The manufacturing process of these products may further introduce chemicals that can pose a potential hazard to a woman's health, including: odor neutralizers, artificial fragrances, artificial colors, or adhesives (which can cause irritation and consequent infection).

Repeated episodic exposure may enhance the risks presented by these toxins present in feminine hygiene products. The average woman will utilize sixteen thousand eight hundred tampons and pads throughout her life. In addition to bodily hazard, conventional feminine hygiene products may also pose a risk to the environment. For example, feminine hygiene products may include materials which are estimated to take as long as six-hundred years to degrade.

### SUMMARY

According to an exemplary embodiment of the present disclosure, an organic material includes a non-permeable bottom layer. The bottom layer includes an upper and a lower surface. At least one core layer is disposed on the upper surface of the bottom layer. The at least one core layer includes a blend of degummed hemp fiber and a super absorbent polymer. An outermost layer includes a permeable composite of organic cotton and hemp fibers disposed on the at least one core layer.

According to an exemplary embodiment of the present disclosure, the bottom layer may include an adhesive surface.

According to an exemplary embodiment of the present disclosure, an adhesive included in the adhesive surface may be cyanoacrylate.

According to an exemplary embodiment of the present disclosure, the lower surface of the bottom layer may be coated with an organic water repellent material.

According to an exemplary embodiment of the present disclosure, the bottom layer may include a hydrophobic material.

According to an exemplary embodiment of the present disclosure, the degummed hemp fiber may be in powder, bast fiber, hurd form or a combination thereof.

According to an exemplary embodiment of the present disclosure, an organic material includes at least one core layer having a cylindrical shape. The at least one core layer includes a blend of degummed hemp fiber, wood pulp, and super absorbent polymer. An outermost layer is disposed on a surface of the at least one core layer. The outermost layer includes a permeable composite of organic cotton mixed and hemp fibers.

According to an exemplary embodiment of the present disclosure, the at least one core layer may include a vegetal cellulose.

According to an exemplary embodiment of the present disclosure, the vegetable cellulose may be arranged in a matrix with the degummed hemp fibers.

According to an exemplary embodiment, the outermost layer may be spun, knitted, or woven.

According to an exemplary embodiment of the present disclosure, the organic cotton may include short staple cotton.

According to an exemplary embodiment of the present disclosure, the short staple cotton may include cotton of the species Gossypium hirsutum.

According to an exemplary embodiment of the present disclosure, the organic cotton may include extra long stable (ELS) cotton.

According to an exemplary embodiment of the present disclosure, the ELS cotton may include cotton of the species Gossypium barbadense.

### BRIEF DESCRIPTION OF THE DRAWINGS

A more complete appreciation of the present disclosure will become more apparent by describing in detail exemplary embodiments thereof with reference to the accompanying drawings, in which:

FIG. 1 is an exploded perspective view illustrating an absorbent organic material according to an exemplary embodiment of the present disclosure.

FIG. 2 is a cross-sectional view of an absorbent organic material according to an exemplary embodiment of the present disclosure.

FIG. 3 is a perspective view of an exemplary feminine hygiene product including the absorbent organic material according to an exemplary embodiment of the present disclosure.

FIG. 4 is a perspective view of an exemplary feminine hygiene product including the absorbent organic material according to an exemplary embodiment of the present disclosure.

FIG. 5 is an exploded perspective view illustrating an absorbent organic material according to an exemplary embodiment of the present disclosure.

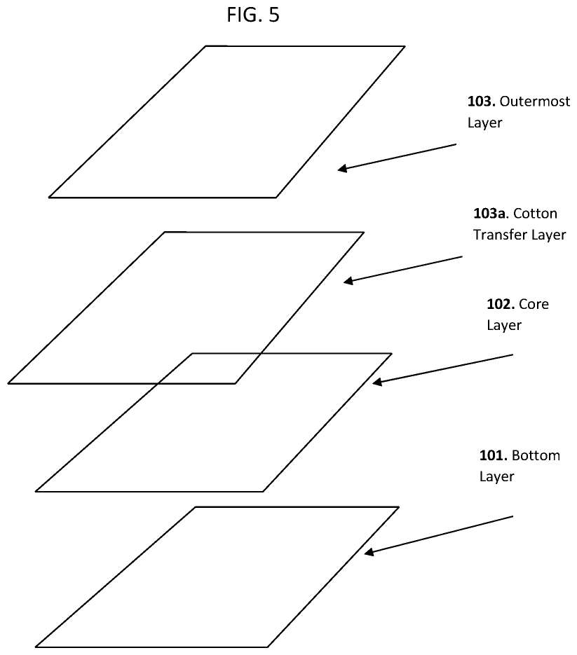

### DETAILED DESCRIPTION

Exemplary embodiments of the present disclosure will be described in more detail with reference to the accompanying drawings. The present disclosure may, however, be embodied in many different forms and should not be construed as being limited to the embodiments set forth herein. Like reference numerals may refer to like elements throughout the specification and drawings.

In the drawings, thicknesses may be illustrated in an enlarged manner in order to clearly describe a plurality of layers and areas. Like reference numbers may be used to denote like elements throughout the specification and drawings. When an element or layer is referred to as being “on”, “engaged to”, “connected to” or “coupled to” another element or layer, it may be directly on, engaged, connected or coupled to the other element or layer, or intervening elements or layers may be present.

The spatially relative terms “below”, “beneath”, “lower”, “above”, “upper”, and the like, may be used herein to describe the relationship between an element or component and another element or component as illustrated in the drawings. It will be understood that the spatially relative terms may encompass different orientations of the device in use or operation, in addition to the orientation depicted in the drawings. The device may also be oriented in the other direction, and thus the spatially relative terms may be interpreted differently depending on the orientations.

It will be understood that, although the terms “first,” “second,” “third,” and the like may be used herein to describe various elements, these elements should not be limited by these terms.

Terms and names of elements used herein may differ from the actual names of products.

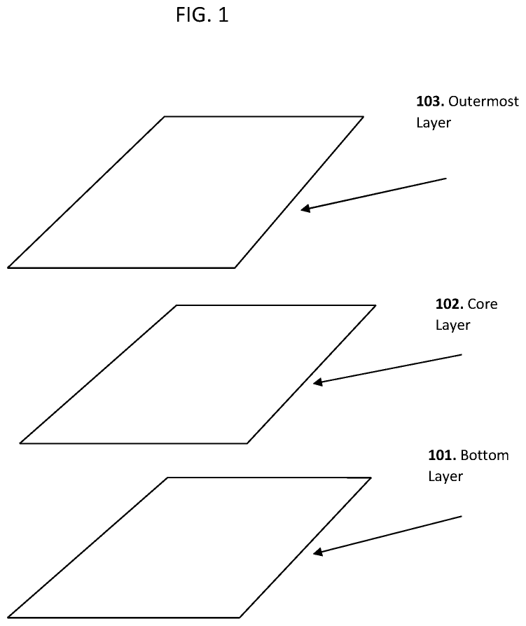

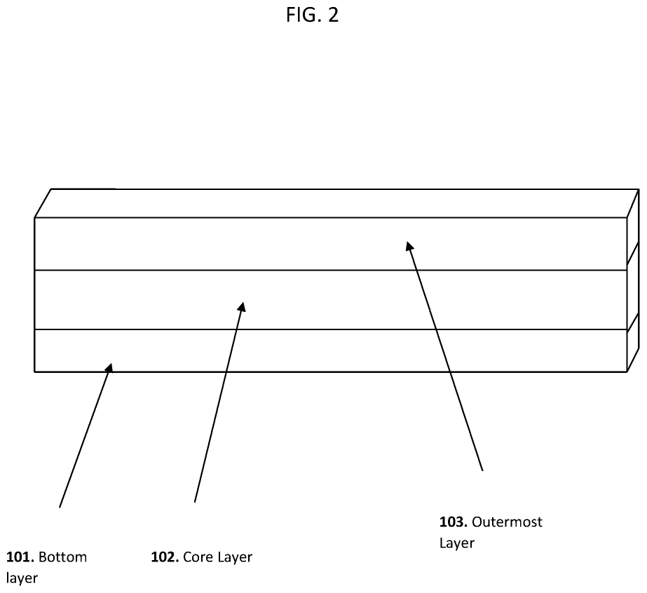

An absorbent organic material according to an exemplary embodiment of the present disclosure will be described in more detail below with reference to FIGS. 1 and 2.

FIG. 1 is an exploded perspective view illustrating an absorbent organic material according to an exemplary embodiment of the present disclosure. FIG. 2 is a cross-sectional view of an absorbent organic material according to an exemplary embodiment of the present disclosure.

Referring to FIGS. 1 and 2, according to an exemplary embodiment of the present disclosure, an organic material (e.g., an absorbent organic material) may include a non-permeable bottom layer 101. The bottom layer 101 may include an upper and a lower surface. At least one core layer 102 may be disposed on the upper surface of the bottom layer 101. The at least one core layer 102 may include a blend of degummed hemp fiber, wood pulp, and a super absorbent polymer. An outermost layer 103 may include a permeable composite of organic cotton and hemp fibers disposed on the at least one core layer.

As an example, the outermost layer 103 may be a top sheet including an all-natural blend of cotton and hemp. The core layer 102 may include hemp pulp (e.g., hemp ‘fluff pulp’) and super absorbent polymers. The hemp pulp may include fibers having a length of from about 1 to about 3 inches. A cotton transfer layer 103a may be positioned between the outermost layer 103 and the core layer 102. The cotton transfer layer 103a increases a transfer of fluid or moisture from the outermost layer 103 to the core layer 102.

As an example, the absorbent organic material may be individually packaged (e.g., as a single use sanitary napkin), and may be included in feminine hygiene products, such as tampons or pads, as discussed below in more detail.

According to an exemplary embodiment of the present disclosure, the bottom layer 101 may include an adhesive surface. As an example, an adhesive included in the adhesive surface may be cyanoacrylate; however, exemplary embodiments of the present disclosure are not limited thereto. The adhesive surface may face the at least one core layer.

According to an exemplary embodiment of the present disclosure, the lower surface of the bottom layer 101 may be coated with an organic water repellent material. Thus, a bottom surface of the bottom layer 101 may be substantially water proof, while layer positioned above the bottom surface of the bottom layer 101 may have varying to degrees of absorbency. This may prevent absorbed fluids from passing through the bottom layer 101 of the absorbent organic material.

As an example, the bottom layer may include a hydrophobic material. For example, the hydrophobic material may include a paraffin wax.

According to an exemplary embodiment of the present disclosure, the bottom layer 101 may include a flexible plastic material, such as a relatively thin plastic material. The bottom layer 101 may prevent leakage through the bottom layer 101 of the absorbent organic material.

According to an exemplary embodiment of the present disclosure, the degummed hemp fiber may be in powder, bast fiber, hurd form, or a combination thereof.

According to an exemplary embodiment of the present disclosure, the at least one core layer 102 may include a vegetal cellulose. According to an exemplary embodiment of the present disclosure, the vegetable cellulose may be arranged in a matrix with the degummed hemp fibers.

According to an exemplary embodiment, the at least one core layer 102 and/or the outermost layer 103 may be spun, knitted, or woven. Alternatively, the outermost layer 103 may be an unwoven layer.

According to an exemplary embodiment of the present disclosure, the super absorbent polymer may be a polymer capable of absorbing at least 40 times its weight in liquid.

According to an exemplary embodiment of the present disclosure, the organic cotton may include short staple cotton. According to an exemplary embodiment of the present disclosure, the short staple cotton may include cotton of the species Gossypium hirsutum.

According to an exemplary embodiment of the present disclosure, the organic cotton may include extra long stable (ELS) cotton. According to an exemplary embodiment of the present disclosure, the ELS cotton may include cotton of the species Gossypium barbadense.

As discussed below in more detail, the absorbent organic material may be included in a feminine hygiene product, such as a pad, a tampon or a sanitary napkin, such as a single use (e.g., disposable sanitary napkin).

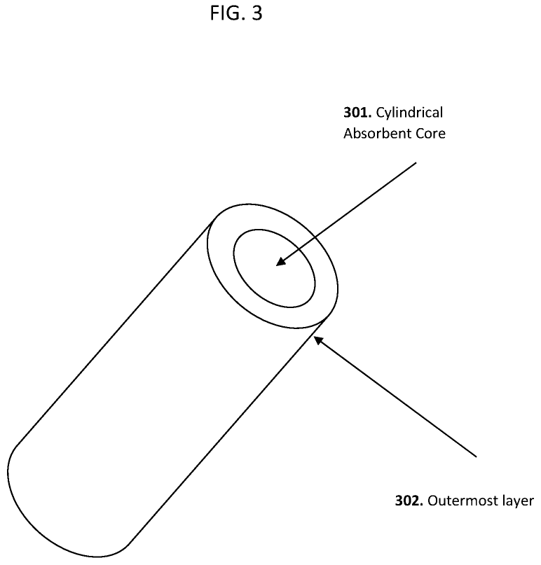

An absorbent organic material according to an exemplary embodiment of the present disclosure will be described in more detail below with reference to FIG. 3.

FIG. 3 is a perspective view of an exemplary feminine hygiene product including the absorbent organic material according to an exemplary embodiment of the present disclosure. For example, the feminine hygiene product according to an exemplary embodiment of the present disclosure described with reference to FIG. 3 may be applicable to a tampon.

Referring to FIG. 3, according to an exemplary embodiment of the present disclosure, a feminine hygiene product including the absorbent organic material described herein may include at least one core layer 301 having a cylindrical shape. The at least one core layer 301 may includes a blend of degummed hemp fiber, wood pulp, and super absorbent polymer. An outermost layer 302 may be disposed on a surface of the at least one core layer 301. The outermost layer 302 may include a permeable composite of organic cotton mixed and hemp fibers. For example, the organic cotton and the hemp fibers may be in an unwoven form in the outermost layer 302.

A tampon according to an exemplary embodiment of the present disclosure may include the substantially cylindrical absorbent core 301 including the degummed hemp fiber, wood pulp, super absorbent polymer, and optionally vegetal cellulose to form a sponge like matrix. The degummed hemp fiber of the cylindrical core may be in the form of hurd, powder, fiber bast, or a mixture thereof.

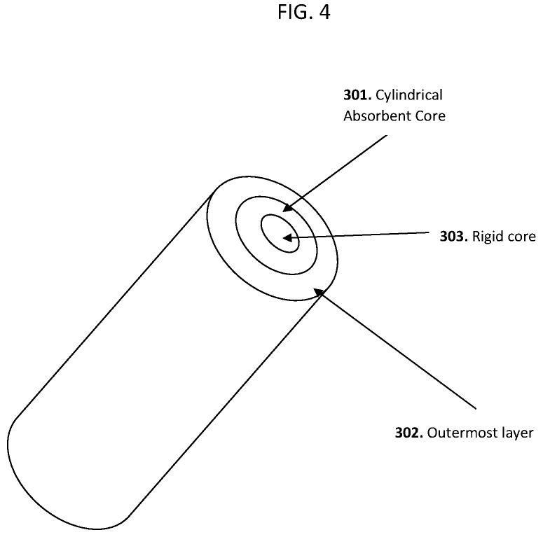

FIG. 4 is a perspective view of an exemplary feminine hygiene product including the absorbent organic material according to an exemplary embodiment of the present disclosure. For example, the feminine hygiene product according to an exemplary embodiment of the present disclosure described with reference to FIG. 4 may be applicable to a tampon including a relatively rigid core. Components that are the same or substantially the same as those described above with reference to FIG. 3 may be omitted below with reference to FIG. 4. Each of the elements according to an exemplary embodiment of the present disclosure described above with reference to FIG. 3 are similarly available to an exemplary embodiment of the present disclosure described below in more detail with reference to FIG. 4.

Referring to FIG. 4, according to an exemplary embodiment of the present disclosure, a feminine hygiene product including the absorbent organic material described herein may include at least one core layer 301 having a cylindrical shape. The at least one core layer 301 may be disposed on a relatively rigid inner core 303. The inner core 303 may include for example, a relatively rigid organic paper product, such as an organic cardboard.

The at least one core layer 301 may include a blend of degummed hemp fiber, wood pulp, and super absorbent polymer. An outermost layer 302 may be disposed on a surface of the at least one core layer 301. The outermost layer 302 may include a permeable composite of organic cotton mixed and hemp fibers. For example, the organic cotton and the hemp fibers may be in an unwoven form in the outermost layer 302.

A tampon according to an exemplary embodiment of the present disclosure may include the substantially cylindrical absorbent core 301 including the degummed hemp fiber, wood pulp, super absorbent polymer, and optionally vegetal cellulose to form a sponge like matrix. The degummed hemp fiber of the cylindrical core may be in the form of hurd, powder, fiber bast, or a mixture thereof.

Having described exemplary embodiments of the present disclosure, it is further noted that it is readily apparent to those of ordinary skill in the art that various modifications may be made without departing from the spirit and scope of the present disclosure.

What is claimed is:

1. An organic material, comprising:
a bottom layer comprising a non-permeable organic substrate having a lower and upper surface;
at least one core layer disposed on the upper surface of the first layer including a blend of degummed hemp fiber and super absorbent polymer; and
an outermost layer comprising a permeable composite of organic cotton and hemp fibers disposed on the core layer.

2. The organic material of claim 1, wherein the bottom layer has at least one adhesive surface.

3. The organic material of claim 2, wherein the adhesive used is cyanoacrylate.

4. The organic material of claim 1, wherein the upper surface of the bottom layer is coated with an organic water repellent.

5. The organic material of claim 1, wherein the non-permeable organic substrate of the bottom layer includes a hydrophobic material.

6. The organic material of claim 1, further comprising a cotton transfer layer between the outermost layer and the at least one core layer.

7. The organic material of claim 1, wherein the organic material is formed into an organic pad.

8. An organic material comprising:
at least one core layer that is substantially cylindrical including a blend of degummed hemp fiber and super absorbent polymer; and
an outermost layer disposed on the surface of the substantially cylindrical core layer comprising a permeable composite of organic cotton and hemp fibers.

9. The organic material of claim 8, further comprising a cotton transfer layer between the at least one core layer and the outermost layer.

10. The organic material of claim 8, wherein the outermost layer is woven, spun, knitted, or bound by an organic filler.
